(19) United States
(12) Patent Application Publication
(10) Pub. No.: US 2024/0285715 A1
(43) Pub. Date: Aug. 29, 2024

(54) PROCESS FOR PREPARATION OF MITRAGYNINE ENRICHED EXTRACT FROM MITRAGYNA SPECIOSA

(71) Applicant: LUCKY 4U EXIMS, Tirupati (IN)

(72) Inventors: Surya Pavan Kumar Avva, Tirupati (IN); V.S.R. Suresh Mamidipalli, Tirupati (IN)

(21) Appl. No.: 18/311,634

(22) Filed: May 3, 2023

(30) Foreign Application Priority Data
Feb. 17, 2023 (IN) 202341010687

(51) Int. Cl. A61K 36/74; A61K 31/4375

(52) U.S. Cl. CPC A61K 36/74; A61K 31/4375; A61K 2236/13; A61K 2236/17; A61K 2236/331; A61K 2236/35; A61K 2236/39; A61K 2236/55

(57) ABSTRACT

The present invention discloses an improved process for preparing enriched extracts containing Mitragynine from the plant Mitragyna speciosa. The present invention discloses simple processes for preparing such enriched extracts of Mitragynine from Mitragyna speciosa using simple and commonly available solvents and inexpensive techniques. More particularly, the present invention relates to preparation of Mitragynine enriched extract using alkaline water solution.

### CROSS-REFERENCE TO RELATED APPLICATION

This application claims foreign priority benefits under 35 U.S.C. § 119 from Indian Patent Application No. 202341010687, filed Feb. 17, 2023, the content of which is hereby incorporated by reference in its entirety.

### TECHNICAL FIELD

The present invention relates to an improved process for preparing enriched extracts containing Mitragynine from the plant Mitragyna speciosa. The present invention particularly relates to simple processes for preparing such enriched extracts of Mitragynine from Mitragyna speciosa using simple and commonly available solvents and inexpensive techniques. More particularly, present invention relates to preparation of Mitragynine enriched extract using alkalined water solution.

### BACKGROUND

Mitragyna speciosa, is a 4-16 metres high tropical evergreen tree belonging to the Rubiaceae family, found in both Asia and Africa. It is commonly known as kratom. Kratom has opioid properties and some stimulant-like effects. Traditionally, fresh or dried kratom leaves were chewed or made into tea or smoked. At a low dose, kratom is used to reduce fatigue during long working hours. At high dosages it can have sedative-narcotic effects. It is used in traditional medicine and as an opium substitute. Over 40 structurally related alkaloids can be isolated from various parts of the plant.

Mitragynine is the main indole alkaloids commonly found in the leaves of Mitragyna speciosa. It was first isolated in 1921 and its chemical structure was fully elucidated in 1964. Mitragynine is chemically known as methyl (E)-2-[(2S,3S,12bS)-3-ethyl-8-methoxy-1,2,3,4,6,7,12,12b-octahydroindolo[2,3-a] quinolizin-2-yl]-3-methoxyprop-2-enoate. The chemical structure of Mitragynine is represented as:

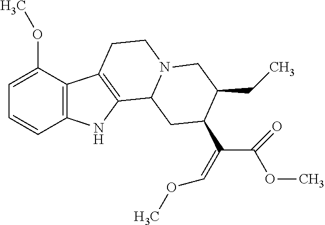 [SMILES: CC[C@@H]1CN2CCc3c([nH]c4cccc(OC)c34)C2C[C@@H]1/C(=C\OC)C(=O)OC]

Mitragynine possesses several pharmacological properties such as antinociceptive and anti-inflammatory. Pharmacologically, Mitragynine is one of the most widely supplied new psychoactive substances used for medical purposes as a pain reliever, and in the treatment of diarrhea, fever, diabetes, and hypertension.

Traditionally known methods used for extraction of compounds from plants includes maceration, percolation, reflux, and Soxhlet extraction. However, these methods require longer time to complete and uses huge quantity of solvents. These solvents are hazardous and toxic solvents which are potentially harmful to health and the environment. The toxicity of the solvents, the degradation of the compounds, consumption of time, total yield and the selectivity of the process are major considerable setbacks in the traditionally known extraction processes (Easmin et. al., Bioactive compounds and advanced processing technology: Phaleria macrocarpa (sheff.) Boerl, a review, Journal of Chemical Technology & Biotechnology 90(6), December 2014).

Presently there are several methods available for the extraction and isolation of Mitragynine from Mitragyna speciosa leaves. These extraction and isolation methods available in the market have their own disadvantages.

Peter J. Houghton et. al., Alkaloids from Mitragyna speciosa, Phytochemistry, Volume 30, Issue 1, Pages 347-350, 1991, discloses extraction of Mitragynine from M. speciosa Leaves comprising macerating M. speciosa Leaves with cold MeOH for 3 days. The filtrate was added with 10% acetic acid and partitioned with 2×50 ml petrol (boiling point 60-8° C.). The water layer was made alkaline by adding sodium carbonate and extracted with 3×30 ml chloroform. The chloroform layer was then partitioned with water.

Soxhlet extraction using methanol is commonly used technique for the extraction of Mitragynine from M. speciosa leaves. Goh Teik Beng et. al., A simple and cost-effective isolation and purification protocol of Mitragynine from Mitragyna speciosa korth (ketum) leaves, Malaysian Journal of Analytical Sciences 15(1):54-60, January 2011, discloses extraction of Mitragynine from Mitragyna speciosa Korth leaves by Soxhlet method using petroleum ether solvent for 8 hours at a temperature of 40-60° C.

S N Harizal et. al., Acute toxicity study of the standardized methanolic extract of Mitragyna speciosa Korth in rodent, J Ethnopharmacol, 131(2):404-9, Sep. 15, 2010, discloses Extraction by Soxhlet method using MeOH solvent for 4 hours at 60° C.

However, these methods are time consuming, such as maceration takes up to 3-4 days or Soxhlet method takes 1-2 days. Furthermore, these involve large amounts of organic solvents such as methanol which are hazardous for health and environment, difficult to remove the solvent completely, costly, require high purity of solvent and low extraction selectivity. Moreover, Soxhlet involves high temperatures in extracting the plant materials, which may cause degradation of some compounds during the extraction.

Although the prior arts disclose various techniques and methods for extraction of Mitragynine, these processes have setback as it results in toxicity of the solvents, the degradation of the compounds, consumption of time, or involves use of expensive instruments.

By aforementioned facts, there exists a need for development of a faster, more economical, simple and commercially significant process for extraction of Mitragynine.

### SUMMARY

The primary object of the present invention is to provide an improved process for preparing enriched extracts containing Mitragynine from the plant Mitragyna speciosa.

Another object of the invention is to provide simple processes for preparing such enriched extracts of Mitragynine from Mitragyna speciosa using simple and commonly available solvents and inexpensive techniques.

Another object of the invention is to provide a process for preparation of Mitragynine enriched extract using alkalined water solution.

Another object of the invention is to provide an improved process resulting in high content Mitragynine extract with higher yield.

Further object of the present invention is to provide a high content Mitragynine extract.

Yet another object of the invention is to meet the drawbacks of the prior art processes with respect to selectivity, efficiency, relatively low cost, and safety.

Present inventors have developed a simple and cost-effective method for the isolation of Mitragynine from Mitragyna speciosa plant by using commonly available solvents and inexpensive techniques. The present method is simpler, faster, more economical and environment friendly.

Accordingly in one aspect, the present invention provides a process for preparation of Mitragynine enriched extract comprising the steps of:
(a) loading small pieces of dried leaves of Mitragyna speciosa plant into the extractor;
(b) circulating alkalined water solution through the dried leaves of step (a) in the extractor;
(c) allowing the mixture of alkalined water solution and dried leaves in step (b) to settle for 15 minutes-3 hours;
(d) draining the alkalined water solution from the extractor leaving the alkalined leaves in the extractor;
(e) washing the alkalined leaves obtained in step (d) in the extractor with protic solvent;
(f) circulating organic solvent in the extractor through the washed alkalined leaves of step (e) forming a solution containing organic solvent and free Mitragynine;
(g) draining the solution containing organic solvent and free Mitragynine of step (f) from the extractor, leaving the spent alkalined leaves in the extractor;
(h) concentrating the drained solution of step (g) followed by drying to obtain Mitragynine enriched extract;
(i) optionally performing steps (f), (g) and (h) multiple times by circulating organic solvent through the spent alkalined leaves of earlier stage in the extractor to obtain the solution containing organic solvent and free Mitragynine followed by draining the solution, concentrating and drying, leaving the spent alkalined leaves in the extractor; and
(j) subjecting the spent alkalined leaves of step (g) or (i) to stripping until 90-99% of solvent is recovered.

The alkalined water solution employed in step (b) of the present process is prepared by adding Alkali to water.

The alkali is selected from the group comprising of sodium hydroxide, potassium hydroxide, ammonium hydroxide, or calcium hydroxide, preferably calcium hydroxide.

The alkalined water solution employed in step (b) of the present process is calcium hydroxide solution.

In step (b) of the present process, the circulation is carried out for 20 minutes to 50 minutes at ambient temperature.

In step (c) of the present process, the mixture of alkalined water solution and dried leaves is allowed to settle for 1 hour at ambient temperature.

The protic solvent employed in step (e) of the present process is selected from a group comprising of water, methanol, ethanol, isopropanol, butanol, acetic acid or mixture thereof, preferably the protic solvent is water.

In step (f) of the present process, the circulation is carried out for 5-15 hours at temperature 35-40° C.

The organic solvent employed in present process is selected from a group comprising of methanol, acetone, isopropyl alcohol, ethanol, chloroform, diethyl ether, petroleum sprit, benzene, dichloromethane, or mixture thereof, preferably the organic solvent is dichloromethane.

In step (f) of the present process, the obtained solution comprises dichloromethane and free Mitragynine.

In step (h) of the present process, the drained solution is concentrated via distillation, wherein the organic layer comprising the Mitragynine enriched extract in the form of paste is obtained.

In step (h) of the present process, the concentrated extract is dried in Vacuum Tray Dryer.

In some embodiment of the invention, there is provided a process for preparation of Mitragynine enriched extract, said process comprises the steps of:
(a) loading small pieces of dried leaves of Mitragyna speciosa plant into the extractor;
(b) circulating alkalined water solution through the dried leaves of step (a) in the extractor for 30 minutes at ambient temperature;
(c) allowing the mixture of alkalined water solution and dried leaves in step (b) to settle for 1 hour at ambient temperature;
(d) draining the alkalined water solution from the extractor leaving the alkalined leaves in the extractor;
(e) washing the alkalined leaves obtained in step (d) in the extractor with water;
(f) circulating dichloromethane in the extractor through the washed alkalined leaves of step (e) for 10 hours at temperature 38-40° C., forming a solution containing dichloromethane and free Mitragynine;
(g) draining the solution containing dichloromethane and free Mitragynine of step (f) from the extractor, leaving the spent alkalined leaves in the extractor;
(h) subjecting the drained solution of step (g) to distillation, followed by drying to obtain Mitragynine enriched extract;
(i) again circulating dichloromethane through the spent alkalined leaves of step (g) in the extractor for 8 hours at temperature 38-40° C., to obtain the solution containing dichloromethane and free Mitragynine followed by draining the solution, leaving the spent alkalined leaves in the extractor;
(j) subjecting the drained solution of step (i) to distillation, followed by drying to obtain Mitragynine enriched extract; and
(k) subjecting the spent alkalined leaves of step (i) to stripping until 90-99% of dichloromethane is recovered.

In some embodiment of the invention, there is provided a process for preparation of Mitragynine enriched extract, said process comprises the steps of:
(a) loading small pieces of dried leaves of Mitragyna speciosa plant into the extractor;
(b) circulating alkalined water solution through the dried leaves of step (a) in the extractor for 30 minutes at ambient temperature;
(c) allowing the mixture of alkalined water solution and dried leaves in step (b) to settle for 1 hour at ambient temperature;
(d) draining the alkalined water solution from the extractor leaving the alkalined leaves in the extractor;
(e) washing the alkalined leaves obtained in step (d) in the extractor with water;
(f) circulating dichloromethane in the extractor through the washed alkalined leaves of step (e) for 10 hours at temperature 38-40° C., forming a solution containing dichloromethane and free Mitragynine;
(g) draining the solution containing dichloromethane and free Mitragynine of step (f) from the extractor, leaving the spent alkalined leaves in the extractor;
(h) subjecting the drained solution of step (g) to distillation, followed by drying to obtain Mitragynine enriched extract;
(i) again circulating dichloromethane through the spent alkalined leaves of step (g) in the extractor for 8 hours at temperature 38-40° C., to obtain the solution containing dichloromethane and free Mitragynine followed by draining the solution, leaving the spent alkalined leaves in the extractor;
(j) subjecting the drained solution of step (i) to distillation, followed by drying to obtain Mitragynine enriched extract;
(k) again circulating dichloromethane through the spent alkalined leaves of step (i) in the extractor for 4 hours at temperature 38-40° C., to obtain the solution containing dichloromethane and free Mitragynine followed by draining the solution;
(l) subjecting the drained solution of step (k) to distillation, followed by drying to obtain Mitragynine enriched extract; OR transferring the drained solution of step (k) to the new batch of dried leaves of Mitragyna speciosa plant; and
(m) subjecting the spent alkalined leaves of step (k) to stripping until 90-99% of dichloromethane is recovered.

The quantity of Mitragynine in the obtained extract is 10.85 to 12.00 grams of Mitragynine with respect to 1 kg of dried leaves. The obtained extract contains 28-34% of assay by HPLC method.

The total recovery of Mitragynine in the obtained extract is 95-97% from the leaves.

### DETAILED DESCRIPTION

The following detailed description refers to the accompanying drawing that show, by way of illustration, specific details and embodiments in which the invention may be practised. These embodiments are described in sufficient detail to enable those skilled in the art to practise the invention. Other embodiments may be utilized, and changes may be made without departing from the scope of the invention. The various embodiments are not necessarily mutually exclusive, as some embodiments can be combined with one or more other embodiments to form new embodiments.

Unless defined otherwise, all technical and scientific terms used herein have the same meaning as commonly understood by one of ordinary skill in the art to which the methods belong.

Further the embodiments described herein can be understood more readily by reference to the following detailed description, examples, and drawings. Methods described herein are merely illustrative of the principles of the present invention and are not limited to the specific embodiments presented in the detailed description, examples, and drawings. Numerous modifications and adaptations will be readily apparent to those of skill in the art without departing from the spirit and scope of the invention.

Where a range of values is provided, it is understood that each intervening value between the upper and lower limit of that range and any other stated or intervening value in that stated range, is encompassed within by the methods. The upper and lower limits of these smaller ranges may independently be included in the smaller ranges and are also encompassed within by the methods, subject to any specifically excluded limit in the stated range. Where the stated range includes one or both of the limits, ranges excluding either or both of those included limits are also included in the methods.

The instant invention describes processes for extraction of Mitragynine from the Mitragyna speciosa plant, more particularly from Mitragyna speciosa leaves. Mitragynine, is a naturally occurring indole alkaloid that can be isolated from the leaves of said psychoactive medicinal plant, Mitragyna speciosa. The extraction processes of the present invention are particularly advantageous because the processes results in Mitragynine enriched extract.

The present process for extraction of Mitragynine comprises treatment of Mitragyna speciosa leaves with alkalined water solution to form alkalined leaves, rich in free Mitragynine which is further treated with organic solvent to obtain Mitragynine enriched extract.

The inventors of the present invention have surprisingly found that using simple and commonly available solvents and inexpensive techniques results in highly enriched Mitragynine extract.

The quantity of Mitragynine in the obtained extract is analysed by HPLC and found that the final extract obtained from 1 kilograms of dried leaves comprises approximately 10.85 to 12.00 grams of Mitragynine. The total recovery of Mitragynine is 95-97% from the raw leaves.

The present invention is described hereinafter in more details substantiating various embodiments for better understanding of the invention.

In one aspect of the invention, there is provided a process for preparation of Mitragynine enriched extract comprising the steps of:
(a) loading small pieces of dried leaves of Mitragyna speciosa plant into the extractor;
(b) circulating alkalined water solution through the dried leaves of step (a) in the extractor;
(c) allowing the mixture of alkalined water solution and dried leaves in step (b) to settle;
(d) draining the alkalined water solution from the extractor leaving the alkalined leaves in the extractor;
(e) washing the alkalined leaves obtained in step (d) in the extractor with protic solvent;
(f) circulating organic solvent in the extractor through the washed alkalined leaves step (e) forming a solution containing organic solvent and free Mitragynine;
(g) draining the solution containing organic solvent and free Mitragynine of step (f) from the extractor, leaving the spent alkalined leaves in the extractor;
(h) concentrating the drained solution of step (g) followed by drying to obtain Mitragynine enriched extract;
(i) optionally performing steps (f), (g) and (h) multiple times by circulating organic solvent through the spent alkalined leaves of earlier stage in the extractor to obtain the solution containing organic solvent and free Mitragynine followed by draining the solution, concentrating and drying, leaving the spent alkalined leaves in the extractor; and
(j) subjecting the spent alkalined leaves of step (g) or (i) to stripping until 90-99% of solvent is recovered.

The leaves of Mitragyna speciosa plant from which Mitragynine is isolated as used in the present invention is obtained from Indonesia.

The leaves of Mitragyna speciosa plant used in the present invention is reduced to suitable size followed by drying, before loading it into the extractor as mentioned in step (a) of above-mentioned process.

The leaves are reduced to suitable size using mechanical cutter or any leaf cutting machines.

The leaves are dried by the methods known in the state of art. Drying preserves the quality of herbs by reducing the moisture content, which inhibits the growth of microorganisms and chemical alterations during dried storage.

The most common methods for drying known in the art are sun drying, shade drying, hot air drying, contact drying, infrared drying, freeze-drying, fluidized bed drying, dielectric drying etc. Depending on the nature of the products to be treated, these methods prove to be more or less adapted. Preferably the leaves of the present invention are dried using shade drying technique which has been proved to be most beneficial method for drying herbs in the art.

The obtained small pieces of dried leaves of Mitragyna speciosa plant are subjected to extraction by the process provided by the present invention.

The alkalined water solution employed in step (b) of the present process is prepared by adding Alkali to water.

The Alkali is selected from the group comprising of sodium hydroxide, potassium hydroxide, ammonium hydroxide, or calcium hydroxide.

Preferably the alkali is calcium hydroxide.

Preferably, the alkalined water solution employed in step (b) of the present process is calcium hydroxide solution.

In some embodiment of the invention, the alkalined water solution is added to the extractor comprising small pieces of dried leaves of Mitragyna speciosa plant and allowed to circulate through dried leaves.

The circulation of alkalined water solution through the dried leaves of step (a) in the extractor as mentioned in the step (b) of present process may be carried out for 10 minutes to 1 hour, preferably 20 minutes to 50 minutes, most preferably 30 minutes, at ambient temperature.

In some embodiment of the invention, the mixture of alkalined water solution and dried leaves in step (b) is allowed to settle as mentioned in step (c) of the above-described process for 15 minutes-3 hours. Preferably the mixture of alkalined water solution and dried leaves in step (b) is allowed to settle for 1 hour at ambient temperature.

The addition of alkalined water solution to the dried leaves of Mitragyna speciosa plant is advantageous since the alkalined water solution combines with acids, tannins, chlorophyll and other phenolic substances in the leaves and sets free the Mitragynine salts present in the leaves.

In one embodiment, after allowing the alkalined water solution and dried leaves mixture to settle, the alkalined water solution comprising the other impurities and compounds as mentioned above is drained from the extractor as mentioned in step (d) of above-mentioned process, leaving the alkalined leaves in the extractor which are rich in free Mitragynine salts.

The protic solvent employed in the step (e) may be selected from a group comprising of water, methanol, ethanol, isopropanol, butanol, acetic acid, or mixture thereof. In one preferred embodiment of the invention, the protic solvent employed in the step (e) of the above-described process is water.

In some embodiment of the invention, the organic solvent is added to the extractor and circulated through the washed alkalined leaves of step (e), as mentioned in step (f) of above-mentioned process.

The circulation of organic solvent through the washed alkalined leaves of step (e) in the extractor as mentioned in the step (f) may be performed for 4 hours to 20 hours, preferably 5 hours to 15 hours, most preferably 10 hours, at temperature 35-40° C.

The organic solvent employed in the instant process may be selected from a group comprising of methanol, acetone, isopropyl alcohol, ethanol, chloroform, diethyl ether, petroleum sprit, benzene, dichloromethane, or mixture thereof, or mixture thereof. In one preferred embodiment of the invention, the organic solvent employed in above-described process is dichloromethane.

The resultant solution obtained in step (f) of the present process comprises organic solvent and free Mitragynine. In one preferred embodiment of the invention, the resultant solution obtained in step (f) of the present process comprises dichloromethane and free Mitragynine.

In this step the freed Mitragynine present in the alkalined leaves are dissolved together with other substances in the organic solvent.

The resultant solution comprising organic solvent and free Mitragynine of step (f) is drained through the extractor leaving behind the spent alkalined leaves in the extractor.

In one embodiment of the invention, the resultant solution comprising organic solvent and free Mitragynine obtained in step (g) is concentrated followed by drying to obtain Mitragynine enriched extract, as mentioned in step (h) of above-mentioned process.

The resultant solution comprising organic solvent and free Mitragynine obtained in step (g) is concentrated by the processes as known in the state of art. In one preferred embodiment, the resultant solution comprising organic solvent and free Mitragynine obtained in step (g) is concentrated via distillation wherein the organic layer comprising the Mitragynine enriched extract in the form of paste is obtained.

The Mitragynine enriched extract obtained after concentration is dried by the processes as known in the state of art. Traditionally known methods for drying are sun drying, air drying, contact drying, infrared drying, freeze-drying, fluidized bed drying, dielectric drying etc. In one preferred embodiment, the Mitragynine enriched extract obtained after concentration in step (h) of the present process is loaded into Vacuum Tray Dryer to obtain Mitragynine enriched extract.

The spent alkalined leaves obtained in step (g) of the present process can optionally be treated again with organic solvent followed by draining the resultant solution comprising organic solvent and free Mitragynine, leaving behind the spent alkalined leaves in the extractor defined above.

In another embodiment of the invention, step (i) of the above-mentioned process can be performed multiple time, preferably 2-4 times, more preferably 2 times. In the same embodiment, the temperature at which extraction with organic solvent in step (i) is performed varies between 4 hours to 20 hours.

In one alternate embodiment the resultant solution comprising organic solvent and free Mitragynine after three circulations of organic solvent as mentioned in step (i) of the present process may be transferred to the new batch of alkalined leaves.

In some embodiment of the invention there is provided a process for preparation of Mitragynine enriched extract comprising the steps of:
(a) loading small pieces of dried leaves of Mitragyna speciosa plant into the extractor;
(b) circulating alkalined water solution through the dried leaves of step (a) in the extractor for 30 minutes at ambient temperature;
(c) allowing the mixture of alkalined water solution and dried leaves in step (b) to settle for 1 hour at ambient temperature;
(d) draining the alkalined water solution from the extractor leaving the alkalined leaves in the extractor;
(e) washing the alkalined leaves obtained in step (d) in the extractor with water;
(f) circulating dichloromethane in the extractor through the washed alkalined leaves of step (e) for 10 hours at temperature 38-40° C., forming a solution containing dichloromethane and free Mitragynine;
(g) draining the solution containing dichloromethane and free Mitragynine of step (f) from the extractor, leaving the spent alkalined leaves in the extractor;
(h) subjecting the drained solution of step (g) to distillation, followed by drying to obtain Mitragynine enriched extract;
(i) again circulating dichloromethane through the spent alkalined leaves of step (g) in the extractor for 8 hours at temperature 38-40° C., to obtain the solution containing dichloromethane and free Mitragynine followed by draining the solution, leaving the spent alkalined leaves in the extractor;
(j) subjecting the drained solution of step (i) to distillation, followed by drying to obtain Mitragynine enriched extract; and
(k) subjecting the spent alkalined leaves of step (i) to stripping until 90-99% of dichloromethane is recovered.

In some embodiment of the invention there is provided a process for preparation of Mitragynine enriched extract comprising the steps of:
(a) loading small pieces of dried leaves of Mitragyna speciosa plant into the extractor;
(b) circulating alkalined water solution through the dried leaves of step (a) in the extractor for 30 minutes at ambient temperature;
(c) allowing the mixture of alkalined water solution and dried leaves in step (b) to settle for 1 hour at ambient temperature;
(d) draining the alkalined water solution from the extractor leaving the alkalined leaves in the extractor;
(e) washing the alkalined leaves obtained in step (d) in the extractor with water;
(f) circulating dichloromethane in the extractor through the washed alkalined leaves of step (e) for 10 hours at temperature 38-40° C., forming a solution containing dichloromethane and free Mitragynine;
(g) draining the solution containing dichloromethane and free Mitragynine of step (f) from the extractor, leaving the spent alkalined leaves in the extractor;
(h) subjecting the drained solution of step (g) to distillation, followed by drying to obtain Mitragynine enriched extract;
(i) again circulating dichloromethane through the spent alkalined leaves of step (g) in the extractor for 8 hours at temperature 38-40° C., to obtain the solution containing dichloromethane and free Mitragynine followed by draining the solution, leaving the spent alkalined leaves in the extractor;
(j) subjecting the drained solution of step (i) to distillation, followed by drying to obtain Mitragynine enriched extract;
(k) again circulating dichloromethane through the spent alkalined leaves of step (i) in the extractor for 4 hours at temperature 38-40° C., to obtain the solution containing dichloromethane and free Mitragynine followed by draining the solution;
(l) subjecting the drained solution of step (k) to distillation, followed by drying to obtain Mitragynine enriched extract; OR transferring the drained solution of step (k) to the new batch of dried leaves of Mitragyna speciosa plant; and
(m) subjecting the spent alkalined leaves of step (k) to stripping until 90-99% of dichloromethane is recovered.

The obtained extract contains 28-34% of assay by HPLC method. The yield obtained from 1 kg of leaves is approximately 90-95%.

Certain specific aspect and embodiment of the present invention will be explained in detail with reference to the following examples, which are provided only for purposes of illustration and should not be construed as limiting the scope of the invention in any manner.

### EXAMPLES

The leaves of Mitragyna speciosa plant from which Mitragynine is isolated as used in the present invention is obtained from Indonesia.

### Example 1: Single-Stage Extraction of Mitragynine

The leaves of Mitragyna speciosa plant were washed to remove the dirt and other impurities. The washed leaves were reduced to suitable size by using leaf cutting machine, followed by drying through shade drying technique. The obtained small pieces of dried leaves of Mitragyna Speciosa plant (600 Kgs) were added to the cleaned Extractor.

Alkalined water solution was prepared by adding 5 Kgs of Calcium Hydroxide in 3000 Litres of water. 3000 Litres of said alkaline water solution was added to the extractor comprising small pieces of dried leaves of Mitragyna speciosa plant and allowed to circulate through dried leaves for 30 minutes at ambient temperature. Following the circulation, the mixture of alkalined water solution and dried leaves was allowed to settle for 1 hour at ambient temperature.

After settling, the alkalined water solution comprising the other impurities and compounds such as acids, tannins, chlorophyll and other phenolic substances in the leaves was drained from the extractor, leaving the alkalined leaves in the extractor which was rich in free Mitragynine. The alkalined leaves were further washed with 2000 Litres of fresh water.

Dichloromethane (4000 Litres) was loaded in the extractor and circulated through the washed alkalined leaves for 10 hours at temperature 38-40° C. The resultant dark green coloured solution comprising dichloromethane and free Mitragynine was drained through the reactor leaving behind the spent alkalined leaves in the extractor.

The resultant dark green coloured solution comprising dichloromethane and free Mitragynine as drained from the extractor was concentrated via distillation wherein the organic layer comprising the Mitragynine enriched extract in the form of paste was obtained. The said paste was loaded into Vacuum Tray Dryer to obtain Mitragynine enriched extract.

Finally, the spent alkalined leaves in the extractor were subjected to stripping until 95% of dichloromethane was recovered.

The quantity of Mitragynine in the obtained extract was analysed by HPLC and was found that the obtained extract contains 28% assay through single-stage extraction process.

Yield: 14-15 kgs (70%-75% w/w)

### Example 2: Two-Stage Extraction of Mitragynine

The small pieces of dried leaves of Mitragyna speciosa plant (600 Kgs) as defined in above example 1 were added to the cleaned Extractor.

Alkalined water solution was prepared by adding 5 Kgs of Calcium Hydroxide in 3000 Litres of water. 3000 Litres of said alkaline water solution was added to the extractor comprising small pieces of dried leaves of Mitragyna speciosa plant and allowed to circulate through dried leaves for 30 minutes at ambient temperature. Following the circulation, the mixture of alkalined water solution and dried leaves was allowed to settle for 1 hour at ambient temperature.

After settling, the alkalined water solution comprising the other impurities and compounds such as acids, tannins, chlorophyll and other phenolic substances in the leaves was drained from the extractor, leaving the alkalined leaves in the extractor which was rich in free Mitragynine. The alkalined leaves were further washed with 2000 Litres of fresh water.

Dichloromethane (4000 Litres) (1^{st} wash) was loaded in the extractor and circulated through the washed alkalined leaves for 10 hours at temperature 38-40° C. The resultant dark green coloured solution comprising dichloromethane and free Mitragynine was drained through the reactor leaving behind the spent alkalined leaves of 1^{st} wash in the extractor.

The resultant dark green coloured solution obtained after 1^{st} wash comprising dichloromethane and free Mitragynine as drained from the extractor was concentrated via distillation wherein the organic layer comprising the Mitragynine enriched extract in the form of paste was obtained. The said paste was loaded into Vacuum Tray Dryer to obtain Mitragynine enriched extract.

After draining the 1^{st} wash, Dichloromethane (3000 Litres) (2^{nd} wash) was loaded in the extractor and circulated through the spent alkalined leaves of 1^{st} wash for 8 hours at temperature 38-40° C. The resultant light green coloured solution comprising dichloromethane and free Mitragynine was drained through the reactor leaving behind the spent alkalined leaves of 2^{nd} wash in the extractor.

The resultant light green coloured solution obtained after 2^{nd} wash comprising dichloromethane and free Mitragynine as drained from the extractor was subjected to concentration via distillation wherein the organic layer comprising the Mitragynine enriched extract in the form of paste was obtained. The said paste was loaded into Vacuum Tray Dryer to obtain Mitragynine enriched extract.

Finally, the spent alkalined leaves in the extractor after 2^{nd} wash were subjected to stripping until 95% of dichloromethane was recovered.

After 1^{st} wash:

The quantity of Mitragynine in the obtained extract was analysed by HPLC and was found that the obtained extract contains 28% assay through 1^{st} wash.

Yield: 14-15 kgs (70%-75% w/w)

After 2^{nd} wash:

The quantity of Mitragynine in the obtained extract was analysed by HPLC and was found that the obtained extract contains 32% assay through 2^{nd} wash.

Yield: 4-5 kgs (10%-15% w/w)

### Example 3: Three-Stage Extraction of Mitragynine

The small pieces of dried leaves of Mitragyna speciosa plant (600 Kgs) as defined in above example 1 were added to the cleaned Extractor.

Alkalined water solution was prepared by adding 5 Kgs of Calcium Hydroxide in 3000 Litres of water. 3000 Litres of said alkaline water solution was added to the extractor comprising small pieces of dried leaves of Mitragyna speciosa plant and allowed to circulate through dried leaves for 30 minutes at ambient temperature. Following the circulation, the mixture of alkalined water solution and dried leaves was allowed to settle for 1 hour at ambient temperature.

After settling, the alkalined water solution comprising the other impurities and compounds such as acids, tannins, chlorophyll and other phenolic substances in the leaves was drained from the extractor, leaving the alkalined leaves in the extractor which was rich in free Mitragynine. The alkalined leaves were further washed with 2000 Litres of fresh water.

Dichloromethane (4000 Litres) (1^{st} wash) was loaded in the extractor and circulated through the washed alkalined leaves for 10 hours at temperature 38-40° C. The resultant dark green coloured solution comprising dichloromethane and free Mitragynine was drained through the reactor leaving behind the spent alkalined leaves of 1^{st} wash in the extractor.

The resultant dark green coloured solution obtained after 1^{st} wash comprising dichloromethane and free Mitragynine as drained from the extractor was concentrated via distillation wherein the organic layer comprising the Mitragynine enriched extract in the form of paste was obtained. The said paste was loaded into Vacuum Tray Dryer to obtain Mitragynine enriched extract.

After draining the 1^{st} wash, Dichloromethane (3000 Litres) (2^{nd} wash) was loaded in the extractor and circulated through the spent alkalined leaves of 1^{st} wash for 8 hours at temperature 38-40° C. The resultant light green coloured solution comprising dichloromethane and free Mitragynine was drained through the reactor leaving behind the spent alkalined leaves of 2^{nd} wash in the extractor.

The resultant light green coloured solution obtained after 2^{nd} wash comprising dichloromethane and free Mitragynine as drained from the extractor was subjected to concentration via distillation wherein the organic layer comprising the Mitragynine enriched extract in the form of paste was obtained. The said paste was loaded into Vacuum Tray Dryer to obtain Mitragynine enriched extract.

After draining the 2^{nd} wash, Dichloromethane (2500 Litres) (3^{rd} wash) was loaded in the extractor and circulated through the spent alkalined leaves of 2^{nd} wash for 4 hours at temperature 38-40° C. The resultant pale green coloured solution comprising dichloromethane and free Mitragynine was drained through the reactor leaving behind the spent alkalined leaves of 3^{rd} wash in the extractor.

The resultant pale green coloured solution obtained after 3^{rd} wash comprising dichloromethane and free Mitragynine as drained from the extractor was subjected to concentration via distillation wherein the organic layer comprising the Mitragynine enriched extract in the form of paste was obtained. The said paste was loaded into Vacuum Tray Dryer to obtain Mitragynine enriched extract.

Alternatively, the resultant pale green coloured solution obtained after 3^{rd} wash is transferred to the 1^{st} wash of new batch of dried leaves.

Finally, the spent alkalined leaves in the extractor after 3^{rd} wash were subjected to stripping until 95% of dichloromethane was recovered.

After 1^{st} wash:

The quantity of Mitragynine in the obtained extract was analysed by HPLC and was found that the obtained extract contains 28% assay through 1^{st} wash.

Yield: 14-15 kgs (70%-75% w/w)

After 2^{nd} wash:

The quantity of Mitragynine in the obtained extract was analysed by HPLC and was found that the obtained extract contains 32% assay through 2^{nd} wash.

Yield: 4-5 kgs (10%-15% w/w)

After 3^{rd} wash:

The quantity of Mitragynine in the obtained extract was analysed by HPLC and was found that the obtained extract contains 34% assay through 3^{rd} wash.

Yield: 1-1.5 kgs (5%-8% w/w)

The obtained extract (after combining extract of 1^{st} wash, 2^{nd} wash, and 3^{rd} wash) contains 28-34% of assay by HPLC method. The yield obtained from 1 kg of leaves was approximately 90-95%.

### Example 4: Mitragynine Enriched Extract

The Mitragynine content in the raw leaves was calculated by HPLC and found to be about 1.00% to 2.00% assay.

The quantity of Mitragynine in the obtained final extract was analysed by HPLC and was found that the extract of 1 kilograms of dried leaves comprises approximately 10.85 to 12.00 grams of Mitragynine. The total recovery of Mitragynine is 95-97% from the raw leaves.

What is claimed is:

1. A process for preparation of Mitragynine enriched extract, comprising the steps of:
(a) loading small pieces of dried leaves of Mitragyna speciosa plant into the extractor;
(b) circulating alkalined water solution through the dried leaves of step (a) in the extractor;
(c) allowing the mixture of alkalined water solution and dried leaves in step (b) to settle for 15 minutes-3 hours;
(d) draining the alkalined water solution from the extractor leaving the alkalined leaves in the extractor;
(e) washing the alkalined leaves obtained in step (d) in the extractor with protic solvent;
(f) circulating organic solvent in the extractor through the washed alkalined leaves of step (e) forming a solution containing organic solvent and free Mitragynine;
(g) draining the solution containing organic solvent and free Mitragynine of step (f) from the extractor, leaving the spent alkalined leaves in the extractor;
(h) concentrating the drained solution of step (g) followed by drying to obtain Mitragynine enriched extract;
(i) optionally performing steps (f), (g) and (h) multiple times by circulating organic solvent through the spent alkalined leaves of earlier stage in the extractor to obtain the solution containing organic solvent and free Mitragynine followed by draining the solution, concentrating and drying, leaving the spent alkalined leaves in the extractor; and
(j) subjecting the spent alkalined leaves of step (g) or (i) to stripping until 90-99% of solvent is recovered.

2. The process as claimed in claim 1, wherein alkalined water solution employed in step (b) is prepared by adding Alkali to water.

3. The process as claimed in claim 2, wherein Alkali is selected from the group comprising of sodium hydroxide, potassium hydroxide, ammonium hydroxide, or calcium hydroxide.

4. The process as claimed in claim 2, wherein Alkali is calcium hydroxide.

5. The process as claimed in claim 1, the alkalined water solution employed in step (b) of the present process is calcium hydroxide solution.

6. The process as claimed in claim 1, wherein in step (b) the circulation is carried out for 20 minutes to 50 minutes at ambient temperature.

7. The process as claimed in claim 1, wherein in step (c) the mixture of alkalined water solution and dried leaves is allowed to settle for 1 hour at ambient temperature.

8. The process as claimed in claim 1, wherein the protic solvent employed in step (e) is selected from a group comprising of water, methanol, ethanol, isopropanol, butanol, acetic acid or mixture thereof.

9. The process as claimed in claim 1, wherein in step (e) the protic solvent is water.

10. The process as claimed in claim 1, wherein in step (f) the circulation is carried out for 5-15 hours at temperature 35-40° C.

11. The process as claimed in claim 1, wherein the organic solvent is selected from a group comprising of methanol, acetone, isopropyl alcohol, ethanol, chloroform, diethyl ether, petroleum sprit, benzene, dichloromethane, or mixture thereof.

12. The process as claimed in claim 1, wherein the organic solvent is dichloromethane.

13. The process as claimed in claim 1, wherein in step (f) the obtained solution comprises dichloromethane and free Mitragynine.

14. The process as claimed in claim 1, wherein in step (h) the drained solution is concentrated via distillation, wherein the organic layer comprising the Mitragynine enriched extract in the form of paste is obtained.

15. The process as claimed in claim 1, wherein in step (h) the concentrated extract is dried in Vacuum Tray Dryer.

16. The process as claimed in claim 1, wherein the quantity of Mitragynine in the obtained extract is 10.85 to 12.00 grams of Mitragynine with respect to 1 kg of dried leaves.

17. The process as claimed in claim 1, wherein the obtained extract contains 28-34% of assay by HPLC method.

18. The process as claimed in claim 1, wherein the total recovery of Mitragynine in the obtained extract is 95-97% from the leaves.

19. A process for preparation of Mitragynine enriched extract comprising the steps of:
(a) loading small pieces of dried leaves of Mitragyna speciosa plant into the extractor;
(b) circulating alkalined water solution through the dried leaves of step (a) in the extractor for 30 minutes at ambient temperature;
(c) allowing the mixture of alkalined water solution and dried leaves in step (b) to settle for 1 hour at ambient temperature;
(d) draining the alkalined water solution from the extractor leaving the alkalined leaves in the extractor;
(e) washing the alkalined leaves obtained in step (d) in the extractor with water;
(f) circulating dichloromethane in the extractor through the washed alkalined leaves of step (e) for 10 hours at temperature 38-40° C., forming a solution containing dichloromethane and free Mitragynine;
(g) draining the solution containing dichloromethane and free Mitragynine of step (f) from the extractor, leaving the spent alkalined leaves in the extractor;
(h) subjecting the drained solution of step (g) to distillation, followed by drying to obtain Mitragynine enriched extract;
(i) again circulating dichloromethane through the spent alkalined leaves of step (g) in the extractor for 8 hours at temperature 38-40° C., to obtain the solution containing dichloromethane and free Mitragynine followed by draining the solution, leaving the spent alkalined leaves in the extractor;
(j) subjecting the drained solution of step (i) to distillation, followed by drying to obtain Mitragynine enriched extract; and
(k) subjecting the spent alkalined leaves of step (i) to stripping until 90-99% of dichloromethane is recovered.

20. The process as claimed in claim 19, wherein the quantity of Mitragynine in the obtained extract is 10.85 to 12.00 grams of Mitragynine with respect to 1 kg of dried leaves.

21. The process as claimed in claim 19, wherein the obtained extract contains 28-34% of assay by HPLC method.

22. The process as claimed in claim 19, wherein the total recovery of Mitragynine in the obtained extract is 95-97% from the leaves.

23. A process for preparation of Mitragynine enriched extract comprising the steps of:
(a) loading small pieces of dried leaves of Mitragyna speciosa plant into the extractor;
(b) circulating alkalined water solution through the dried leaves of step (a) in the extractor for 30 minutes at ambient temperature;
(c) allowing the mixture of alkalined water solution and dried leaves in step (b) to settle for 1 hour at ambient temperature;
(d) draining the alkalined water solution from the extractor leaving the alkalined leaves in the extractor;
(e) washing the alkalined leaves obtained in step (d) in the extractor with water;
(f) circulating dichloromethane in the extractor through the washed alkalined leaves of step (e) for 10 hours at temperature 38-40° C., forming a solution containing dichloromethane and free Mitragynine;
(g) draining the solution containing dichloromethane and free Mitragynine of step (f) from the extractor, leaving the spent alkalined leaves in the extractor;
(h) subjecting the drained solution of step (g) to distillation, followed by drying to obtain Mitragynine enriched extract;
(i) again circulating dichloromethane through the spent alkalined leaves of step (g) in the extractor for 8 hours at temperature 38-40° C., to obtain the solution containing dichloromethane and free Mitragynine followed by draining the solution, leaving the spent alkalined leaves in the extractor;
(j) subjecting the drained solution of step (i) to distillation, followed by drying to obtain Mitragynine enriched extract;
(k) again circulating dichloromethane through the spent alkalined leaves of step (i) in the extractor for 4 hours at temperature 38-40° C., to obtain the solution containing dichloromethane and free Mitragynine followed by draining the solution;
(l) subjecting the drained solution of step (k) to distillation, followed by drying to obtain Mitragynine enriched extract; OR transferring the drained solution of step (k) to the new batch of dried leaves of Mitragyna speciosa plant; and
(m) subjecting the spent alkalined leaves of step (k) to stripping until 90-99% of dichloromethane is recovered.

24. The process as claimed in claim 23, wherein the quantity of Mitragynine in the obtained extract is 10.85 to 12.00 grams of Mitragynine with respect to 1 kg of dried leaves.

25. The process as claimed in claim 23, wherein the obtained extract contains 28-34% of assay by HPLC method.

26. The process as claimed in claim 23, wherein the total recovery of Mitragynine in the obtained extract is 95-97% from the leaves.

27. Mitragynine enriched extract of Mitragyna speciosa containing 28-34% of assay by HPLC method.
